(19) United States
(12) Patent Application Publication
(10) Pub. No.: US 2026/0041853 A1
(43) Pub. Date: Feb. 12, 2026

(54) NEEDLE SHIELD ASSEMBLY FOR A SYRINGE

(71) Applicant: AMGEN INC., Thousand Oaks, CA (US)

(72) Inventors: Robert W. Swift, Fillmore, CA (US); James Nakamura, Monterey Park, CA (US); Michael E. Akers, Thousand Oaks, CA (US); Jerome Olivas, Thousand Oaks, CA (US)

(21) Appl. No.: 19/100,434

(22) PCT Filed: Aug. 16, 2023
(86) PCT No.: PCT/US2023/030325

§ 371 Date: Jan. 31, 2025

Related U.S. Application Data
(60) Provisional application No. 63/399,146, filed on Aug. 18, 2022.

(51) Int. Cl. A61M 5/32

(52) U.S. Cl. CPC A61M 5/3202; A61M 2207/00

(57) ABSTRACT

A needle shield assembly includes a first needle shield at least partially made of a first material and having a proximal end and a distal end, the proximal end of the first needle shield including a cavity; and a second needle shield at least partially made of a second material, the second needle shield mechanically coupled with at least the proximal end of the first needle shield. Additionally, the second material of the second needle shield is harder than the first material of the first needle shield.

### CROSS-REFERENCE TO RELATED APPLICATIONS

Priority is claimed to United States Provisional Ser. No. 63/399,146 , filed Aug. 18, 2022, the entire contents of which are hereby incorporated by reference herein.

### FIELD OF DISCLOSURE

The present disclosure generally relates to syringes, and more particularly, to needle shields for use in conjunction with syringes.

### BACKGROUND

A syringe may contain a drug and include a needle for delivering the drug into, for example, a patient. A needle shield may be provided to maintain sterility of the needle, protect the needle from damage, and/or reduce the likelihood of accidental needle stick injuries. To secure the needle shield to the syringe, the needle shield may be designed to frictionally couple with the syringe. Prior to use of the syringe for delivering the drug, the needle shield may need to be removed from the syringe. In at least some instances, the interface between the syringe and needle shield may create substantial resistance to a force applied to remove the needle shield from the syringe.

To remove the needle shield, a user may grab the needle shield and pull it off with their hands and/or employ a needle shield remover. Certain needle shield removers grab an end of the needle shield and the needle shield remover may provide a grip for the user.

### SUMMARY

Disclosed herein is a needle shield assembly including a first needle shield at least partially made of a first material and having a proximal end and a distal end. The proximal end of the first needle shield includes a cavity. The needle shield assembly further includes a second needle shield at least partially made of a second material, the second needle shield mechanically coupled with at least the proximal end of the first needle shield. Additionally, the second material is harder than the first material.

In some variations, the first needle shield further includes an annular shoulder and the second needle shield is mechanically coupled with the first needle shield by at least partially abutting the shoulder. Additionally or alternatively, the second needle shield further abuts the proximal end of the first needle shield. Furthermore, at least a portion of the second needle shield overlays and/or is radially outward of at least a portion of the annular shoulder of the first needle shield. In some examples, the second needle shield is overmolded on the first needle shield.

In yet other variations, the second needle shield further includes teeth at least partially embedded in the first needle shield. Additionally, the teeth are embedded in the first needle shield at approximately a midpoint between the proximal end and the distal end of the first needle shield. Alternatively, the teeth are embedded in the distal end of the first needle shield.

Disclosed herein is a needle shield assembly including a first needle shield having a proximal end and a distal end. The needle shield assembly further includes a second needle shield having a flexible distal portion and a rigid proximal portion. Additionally, the needle shield assembly includes a cap configured to at least partially surround at least one of the first needle shield and the second needle shield and press the flexible distal portion of the second needle portion against the first needle shield.

In some variations, the second needle shield further comprises teeth coupled with by the flexible distal portion. In some examples, the teeth are at least partially embedded in the first needle shield when the cap at least partially surrounds the second needle shield. Additionally, the teeth may be disposed at a distal end of the flexible distal portion of the second needle shield. Alternatively, the teeth may be disposed adjacent a mechanical connection between the flexible distal portion and the rigid portion of the second needle shield.

In other variations, the first needle shield further includes an annular shoulder and the second needle shield is mechanically coupled with the first needle by at least partially abutting the shoulder. For example, the annular shoulder may be disposed proximate the proximal end of the first needle shield. Additionally or alternatively, the second needle shield abuts the proximal end of the first needle shield.

In yet other variations, the second needle shield includes an annular ridge, the cap includes an annular groove and the annular ridge is configured to engage the annular groove to mechanically couple the second needle shield and the cap. In some examples, the cap is configured to mechanically couple the first needle shield, second needle shield, and cap together. In some such examples, the second needle shield is overmolded on the first needle shield.

Also disclosed herein is a method of assembly including, providing a first needle shield at least partially made of a first material and having a proximal end and a distal end. The method further includes mechanically coupling a second needle shield to the first needle shield, the second needle shield at least partially made of a second material. In some examples, the second material is harder than the first material.

In some variations, the method of assembly includes at least partially inserting the first needle shield and the second needle shield into the cap. Further, the method may include mechanically coupling the cap with the second needle shield. In some examples, mechanically coupling the cap with the second needle shield includes disposing at least one annular ridge of the second needle shield in at least one annular groove formed in the cap.

In other variations, the second needle shield includes a flexible distal portion and the cap presses the flexible distal portion of the second needle shield against the first needle shield. Additionally, the flexible distal portion may comprise at least one tooth and wherein mechanically coupling the cap with the second needle shield comprises pressing the flexible distal portion of the second needle shield against the first needle shield such that the at least one tooth is at least partially embedded within the first needle shield.

In yet other variations, the method includes overmolding the second needle shield on the first needle shield. In some such examples, mechanically coupling the second needle shield to the first needle shield comprises disposing an annular shoulder of the first needle shield at least partially in a pocket of the second needle shield.

### BRIEF DESCRIPTION OF THE DRAWINGS

The present disclosure may be best understood by reference to the following description taken in conjunction with the accompanying drawings, in which:

FIG. 1 is a side view of a first example needle shield assembly made in accordance with the present disclosure.

FIG. 2 is a side view and cut-away of the first example needle shield assembly of FIG. 1.

FIG. 3 is a perspective view of a second example needle shield assembly made in accordance with the present disclosure.

FIG. 4 illustrates an example first step of a manufacturing process for the second example needle shield assembly of FIG. 3.

FIG. 5 illustrates an example second step of a manufacturing process for the second example needle shield assembly of FIG. 3.

FIG. 6 illustrates an example third step of a manufacturing process for the second example needle shield assembly of FIG. 3.

FIG. 7 is a perspective view of a third example needle shield assembly made in accordance with the present disclosure.

FIG. 8 illustrates an example first step of a manufacturing process for the third example needle shield assembly of FIG. 7.

FIG. 9 illustrates an example second step of a manufacturing process for the third example needle shield assembly of FIG. 7.

FIG. 10 illustrates an example third step of a manufacturing process for the third example needle shield assembly of FIG. 7.

The figures depict preferred embodiments for purposes of illustration only and are not to scale. One skilled in the art will readily recognize from the following discussion that alternative embodiments of the systems and methods illustrated herein may be employed without departing from the principles described herein.

### DETAILED DESCRIPTION

Prefilled syringes filled with a drug and other syringes are often provided with a needle shield to maintain sterility of a needle of the syringe, protect this needle from damage, and/or reduce the likelihood of accidental needle stick injuries. The needle shield may be tightly secured to the syringe, which can present a challenge for at least some end users in remove the needle shield from the syringe. In some instances, removing a needle shield from a syringe can require a user to manually apply approximately 45 Newtons (N) or more of force, which is beyond the ability of some users.

A needle shield or syringe made in accordance with the present disclosure is configured to provide a reduced friction interface between the needle shield and the syringe without compromising an ability of the needle shield to, for example, maintain the sterility of a needle of the syringe, protect the needle from damage, and/or prevent accidental needle sticks. As a result, the reduced friction interface may make it easier to remove the needle shield from the syringe as compared to conventional arrangements. In at least some embodiments, the force needed to remove the needle shield is less than or equal to approximately 5 N or less than or equal to approximately 10 N. Furthermore, in at least some embodiments, the needle shield or syringe of the present disclosure reduces friction force between the needle shield and the syringe by reducing the surface area of contact between the syringe and the needle shield.

Furthermore, needle shields tend to deform under the force applied to remove the needle shield from a syringe. For example, the needle shield pulled from a distal end may elongate and narrow about the needle. As a result, the deformed needle shield may clamp onto the needle and further increase the force necessary to remove the needle shield from the syringe. In accordance with the present disclosure, the needle shield is configured to distribute the removal force such that the needle shield does not or is less likely to deform with respect to the needle and/or increase the force necessary to remove the needle shield from the syringe.

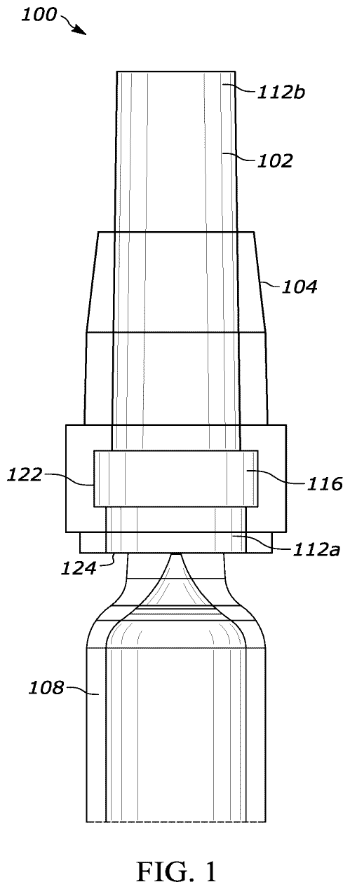

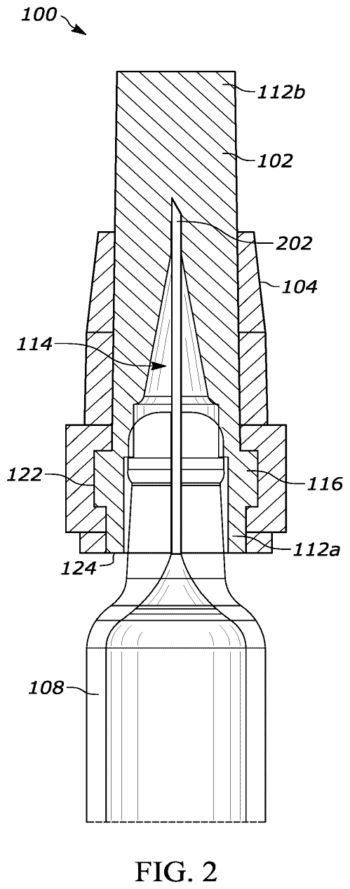

FIGS. 1 and 2 illustrate a first example needle shield assembly 100 made in accordance with the present disclosure. The needle shield assembly 100 includes the first needle shield 102, the second needle shield 104, and the syringe 108. In the illustrated example of FIGS. 1 and 2, the second needle shield 104 is shorter than the first needle shield 102, however in various other examples, the second needle shield 104 is the same length or longer than the first needle shield 102.

The first needle shield 102 is at least partially made of a first material and has a proximal end 112a and a distal end 112b, the proximal end 112a of the first needle shield 102 includes a cavity 114 extending from the proximal end 112a. In some examples, the first needle shield is made entirely of the first material. The cavity 114 is configured to receive and secure a needle, such as needle 202. The needle 202 is shown in FIG. 2, but, for the sake of clarity, may be omitted in other figures. The first needle shield 102 also includes an annular shoulder 116 and the second needle shield is mechanically coupled with the first needle shield by at least partially abutting the shoulder.

The needle shield assembly 100 further includes the second needle 104 shield made of a second material. The second needle shield 104 is mechanically coupled with the first needle shield 102. In the present example, the second needle shield 104 includes a pocket 122 that overlays the annular shoulder 116 and/or is radially outward of at least a portion of the annular shoulder 116. In such examples, the second needle shield 104 transfers axial forces to the first needle shield 102 via the pocket 122 being coupled with the annular shoulder 116. As shown in FIG. 1, the annular shoulder 116 and the pocket 122 are proximate the proximal end 112a of the first needle shield 102. In other examples, the shoulder 116 could be disposed at approximately a midpoint between the proximal end 112a and the distal end 112b or the shoulder 116 could be disposed proximate the distal end 112b of the first shoulder 102. Additionally or alternatively, the second needle shield 104 could abut a proximal face of the first needle shield.

In accordance with the present disclosure the second needle shield 104 is at least partially made of the second material and the first needle shield 102 is made of the first material. In some examples, the second needle shield 104 is entirely made of the second material. In a preferred example, the second material is harder than the first material. As a result, when forces are applied, the second needle shield 104 has limited deformation and can transfer the applied forces to the first needle shield 102. By applying the force from the second needle shield 104 to the first needle shield 102 via the annular shoulder 116, the first needle shield 102 experiences limited deformation and does not clamp onto a needle and increase the forces required to remove the needle shield 102 from the syringe 108. In some examples, the second needle shield 104 is manufactured and overmolded on the first needle shield 102. As used herein, overmolded means that the second needle shield 104 is formed and molded onto the first needle shield 102, rather than being manufactured separately from and subsequently coupled with the first needle shield 102. Alternatively, the second needle shield 104 can be manufactured separately from the first needle shield 102 and later coupled with the first needle shield 102.

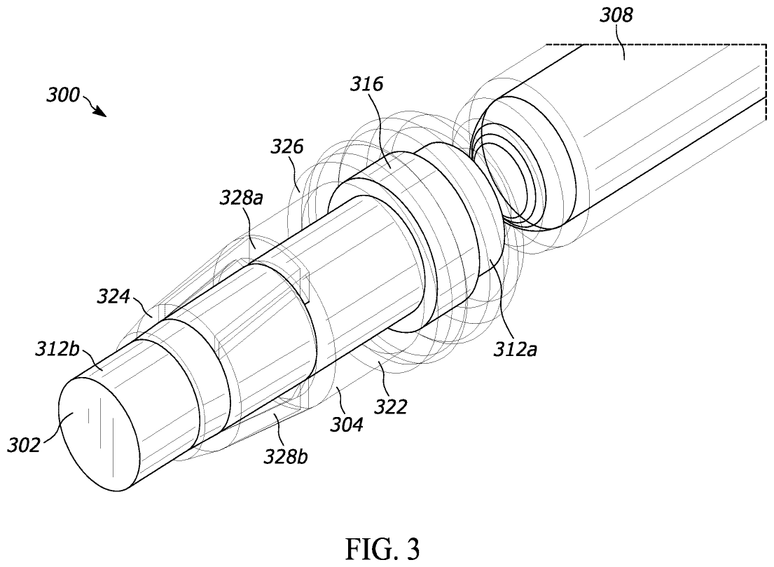

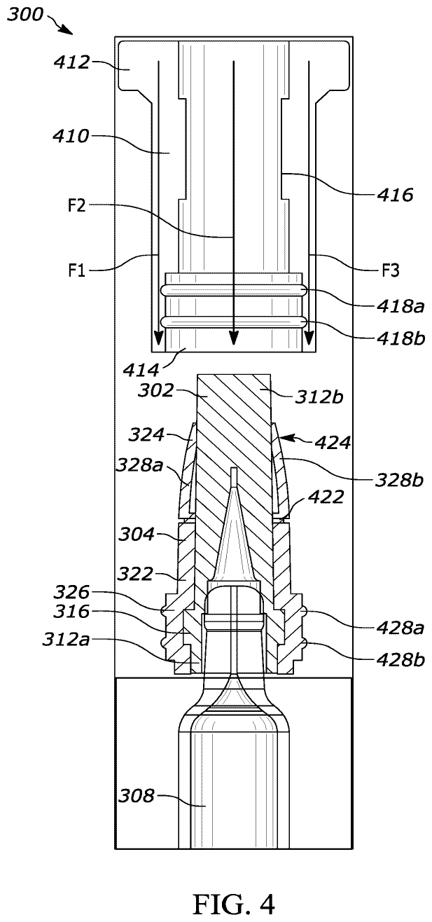

FIG. 3 is a perspective view of a second example needle shield assembly 300 made in accordance with the present disclosure. The needle shield assembly 300 includes a first needle shield 302, a second needle shield 304, a cap 410 (discussed in greater detail in connection with FIGS. 4, 5, and 6), and a syringe 308. In the example illustrated in FIG. 3, the second needle shield 304 is transparent, however, in other examples, the second needle shield 304 may be made of a non-transparent material (e.g., as shown in FIGS. 4, 5, and 6).

The first needle shield 302 includes a proximal end 312a and a distal end 312b. While the first needle shield 302 is generally cylindrical, the first needle shield further includes an annular shoulder 314. As a result, the first needle shield 302 is substantially similar to the first needle shield 102 of FIGS. 1 and 2. In various examples, the first needle shield 302 is made of a first thermoplastic material that is flexible and pliable.

The second needle shield 304 includes a rigid proximal portion 322 and a flexible distal portion 324. The second needle shield further comprises teeth 422 (discussed in more detail below in connection with FIGS. 4, 5, and 6) carried by or coupled with the flexible distal portion 324. In some examples, the second needle shield 304 is manufactured and overmolded on the first needle shield 302. As used herein, overmolded means that the second needle shield 304 is formed and molded onto the first needle shield 302, rather than being manufactured separately from and subsequently coupled with the first needle shield 302. Alternatively, the second needle shield 304 can be manufactured separately from the first needle shield 302 and later coupled with the first needle shield 302.

Additionally, the first needle shield 302 and the second needle shield 304 are mechanically coupled. As illustrated, the first needle shield 302 includes the shoulder 316 and the second needle shield 304 includes a pocket 326. The shoulder 316 and the pocket 326 couple the first needle shield 302 to the second needle shield 304 and inhibit axial movement of the first needle shield 302 relative the second needle shield 304. Also, the annular shoulder 316 is disposed proximate the proximal end 312a of the first needle shield 302. Additionally or alternatively, the second needle shield 304 may abut the proximal end 312a of the first needle shield 302.

The second needle shield 304 further includes at least a first flexible arm 328a and a second flexible arm 328b. In the illustrated example, the second needle shield 304 only includes two flexible arms 328a, 328b. In various other examples, the second needle shield 304 could include more or fewer flexible arms than shown in FIG. 3.

FIG. 4 illustrates an example first step of a manufacturing process for the second example needle shield assembly 300 of FIG. 3. In the illustrated example, the syringe 308 includes the first needle shield 302, the second needle shield 304, and a cap 410 disposed separate from the first and second needle shield 302, 304. As shown by the arrows F1, F2 and F3, the cap 410 is being moved towards the first needle shield 302 and the second needle shield 304. In some examples, the cap 410 may be assembled onto the first and second needle shields 302, 304 prior to disposing the first and second needle shields 302, 304 on the syringe 308.

The cap 410 includes a rim 412, a cavity 414, a sealing surface 416, and first and second annular grooves 418a, 418b. In the present example, the cavity 414 passes through the cap 410, however in other examples, the cavity 414 may only partially pass through the cap 410. Additionally, the cap 410 includes the rim 412, and the rim is configured to assist an end user in pulling the cap off the syringe.

Additionally, as shown in FIG. 4, the teeth 422 are disposed proximate the rigid portion 322. In some examples, the teeth 422 may be disposed proximate or adjacent a mechanical connection 424 between the flexible distal portion 324 and the rigid portion 322 of the second needle shield 304. In the present example, the mechanical connection 424 is disposed proximate the distal end 312b of the first needle shield 302, but in other examples, the mechanical connection 424 could be disposed elsewhere on the second needle shield 304.

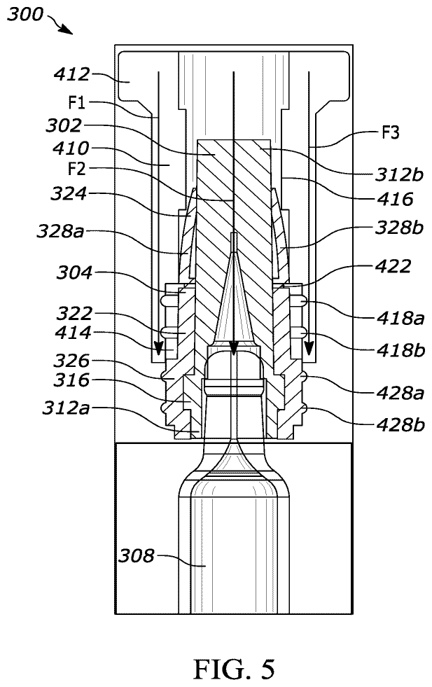

FIG. 5 illustrates an example second step of a manufacturing process for the second example needle shield assembly 300 of FIG. 3. In accordance with the present disclosure, the cap 410 is configured to partially or entirely surround the first needle shield 302 and/or the second needle shield 304. As an example, the cap 410 may at least partially surround the first needle shield 302 and/or the second needle shield 304. As shown in FIG. 5, the cap 410 is about to press the flexible distal portion 324 of the second needle portion onto the first needle shield. Specifically, the sealing surface 416 is about to contact the flexible distal portion 324.

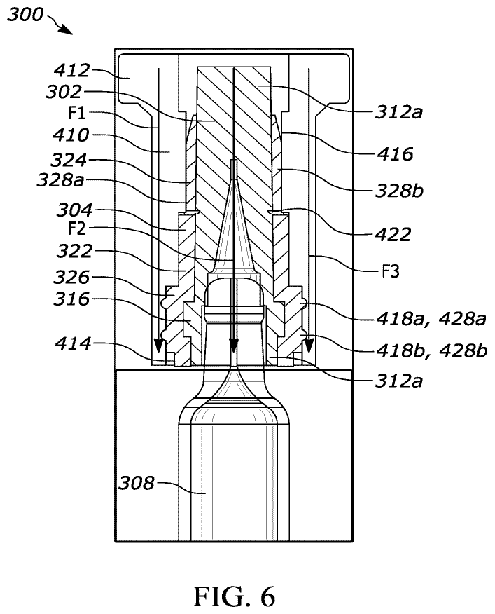

FIG. 6 illustrates an example third step of a manufacturing process for the second example needle shield assembly 300 of FIG. 3. As shown in FIG. 6, the teeth 422 are at least partially embedded into the first needle shield 302 after the cap 410 has surrounded (e.g., enveloped) the second needle shield 304. In the present example, the teeth 422 are embedded in the first needle shield 302 are embedded at approximately a midpoint between the proximal end 312a and the distal end 312b of the first needle shield 302. In various other examples, the teeth 422 may be configured to embed elsewhere in the first needle shield 302, including proximate the proximal end 312a or the distal end 312b.

As shown in FIG. 6, the cap 410 mechanically couples the first needle shield 302, second needle shield 304, and cap 410 together. The cap 410 is mechanically coupled with the second needle shield 304 when the annular grooves 418a, 418b disposed in the cap 410, receive annular ridges 428a, 428b disposed on the second needle shield 304. Additionally, the second needle shield 304 is coupled with the first needle shield 304 because the teeth 422 are embedded into the first needle shield 302 and the annular shoulder 316 coupled with the pocket 326. Accordingly, the first needle shield 302, second needle shield 304, and the cap 410 are mechanically coupled together.

When the first needle shield 302, second needle shield 304, and the cap 410 are coupled together, the force exerted on the rim 412 is distributed through the first needle shield 304 at least at the annular shoulder 316 and via the teeth 422. As a result, the even distribution of the force reduces deformation in the first needle shield 302. Since the first needle shield 302 does not deform, the force needed to remove the needle shield remains relatively low (e.g., approximately 5-10 N or less).

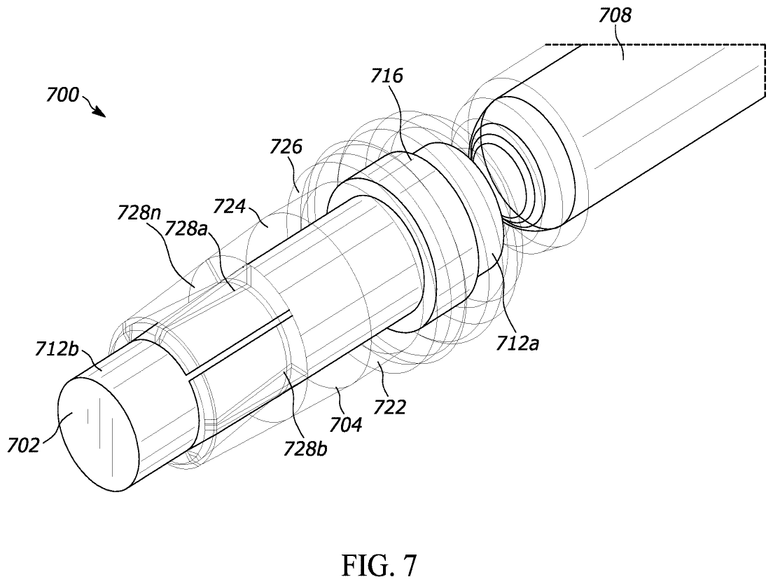

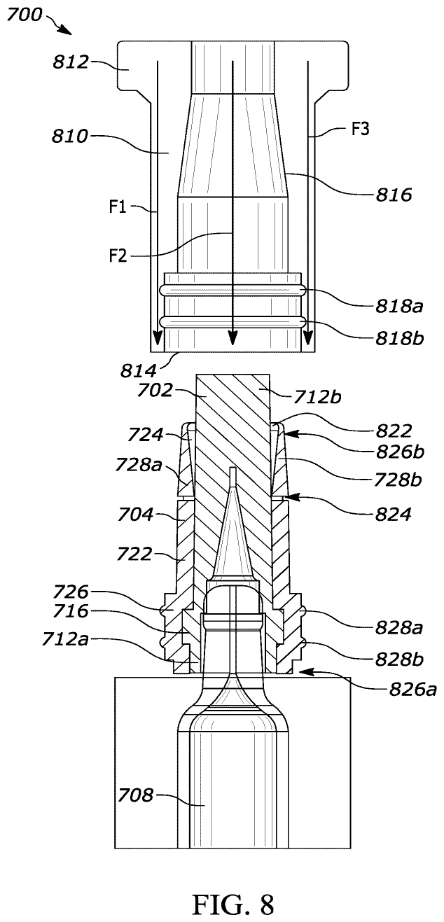

FIG. 7 is a perspective view of a third example needle shield assembly 700 made in accordance with the present disclosure. The needle shield assembly 700 includes a first needle shield 702, a second needle shield 704, a cap 810 (discussed in more detail below in connection with FIGS. 8, 9, and 10), and a syringe 708. In the illustrated example of FIG. 7, the second needle shield 704 is transparent, however, in other examples the second needle shield 704 is made of a non-transparent material (e.g., as shown in FIGS. 8, 9, and 10).

The first needle shield 702 includes a proximal end 712a and a distal end 712b. While the first needle shield 702 is generally cylindrical, the first needle shield further includes an annular shoulder 714. As a result, the first needle shield 702 is substantially similar to the first needle shield 102 of FIGS. 1 and 2 and the first needle shield 302 of FIGS. 3, 4, 5, and 6. In various examples, the first needle shield 702 is made of a first thermoplastic material that is flexible and pliable.

The second needle shield 704 includes a rigid proximal portion 722 and a flexible distal portion 724. The second needle shield further comprises teeth 822 (discussed in greater detail in connection with FIGS. 8, 9, and 10) carried by or coupled with the flexible distal portion 724. In some examples, the second needle shield 704 is manufactured and overmolded on the first needle shield 702. Alternatively, the second needle shield 704 can be manufactured separately from the first needle shield 702 and later coupled with the first needle shield 702.

Additionally, the first needle shield 702 and the second needle shield 704 are mechanically coupled. As illustrated, the first needle shield 702 includes the shoulder 716 and the second needle shield 704 includes a pocket 726. The shoulder 716 and the pocket 726 couple the first needle shield 702 to the second needle shield 704 and inhibit axial movement of the first needle shield 702 relative the second needle shield 704. Also, the annular shoulder 716 is disposed proximate the proximal end 712a of the first needle shield 702. Additionally or alternatively, the second needle shield 704 may abut the proximal end 712a of the first needle shield 702.

The second needle shield 704 further includes at least a first flexible arm 728a and a second flexible arm 728b. In the illustrated example, the second needle shield 704 only includes two flexible arms 728a, 728b. In various other examples, the second needle shield 704 could include more or fewer flexible arms than shown in FIG. 7.

FIG. 8 illustrates an example first step of a manufacturing process for the third example needle shield assembly 700 of FIG. 7. In the illustrated example, the syringe 708 includes the first needle shield 702, the second needle shield 704, and a cap 810 disposed separate from the first and second needle shield 702, 704. As shown by the arrows F1, F2, F3, the cap 810 is being moved towards the first needle shield 702 and the second needle shield 704. In some examples, the cap 810 may be assembled onto the first and second needle shields 702, 704 prior to disposing the first and second needle shields 702, 704 on the syringe 708.

The cap 810 includes a rim 812, a cavity 814, a sealing surface 816, and first and second annular grooves 818a, 818b. In the present example, the cavity 814 passes through the cap 810, however in some examples, the cavity 814 may only partially pass through the cap 810. Additionally, the cap 810 includes the rim 812, and the rim is configured to assist an end user in pulling the cap off the syringe.

Additionally, as shown in FIG. 8, the teeth 822 are disposed proximate the distal end 712b of the first needle shield 702. In some examples, the teeth 822 may be disposed proximate or adjacent a mechanical connection 824 between the flexible distal portion 724 and the rigid portion 722 of the second needle shield 704. In the present example, the mechanical connection 824 is disposed between a proximal end 826a and a distal end 826b of the second needle shield. In other examples, the mechanical connection 824 could be disposed elsewhere on the second needle shield 704.

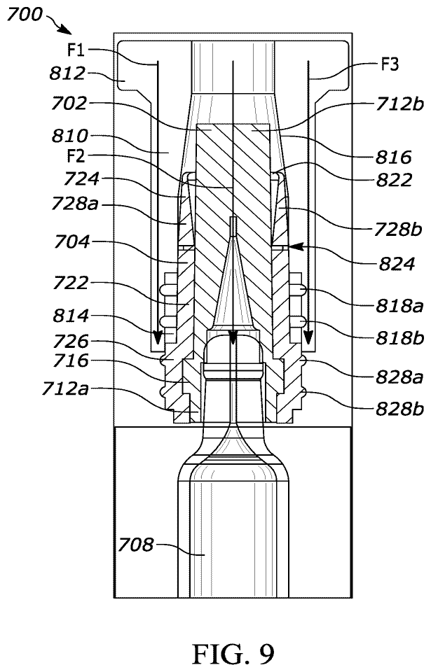

FIG. 9 illustrates an example second step of a manufacturing process for the third example needle shield assembly 700 of FIG. 7. In accordance with the present disclosure, the cap 810 is configured to partially or entirely surround the first needle shield 702 and/or the second needle shield 704. As an example, the cap 810 may at least partially surround the first needle shield 702 and/or the second needle shield 704. As shown in FIG. 9, the cap 810 is about to press the flexible distal portion 724 of the second needle portion onto the first needle shield 700. Specifically, the sealing surface 816 is about to contact the flexible distal portion 724.

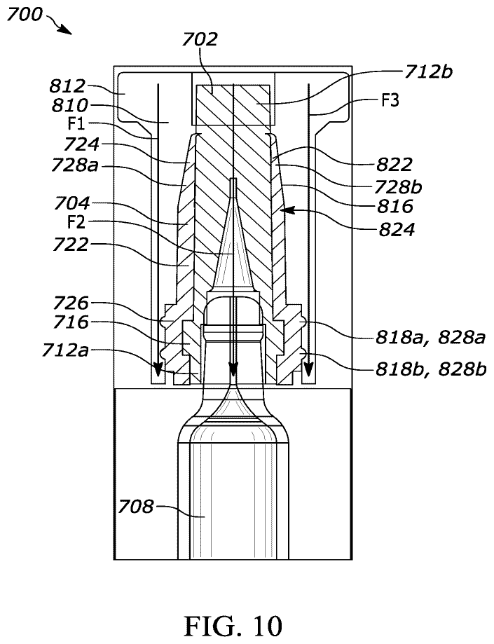

FIG. 10 illustrates an example third step of a manufacturing process for the third example needle shield assembly 700 of FIG. 7. As shown in FIG. 10, the teeth 822 are embedded into the first needle shield 702 after the cap 810 has surrounded the second needle shield 704. In the present example, the teeth 822 are embedded in the first needle shield 702 are embedded at approximately a midpoint between the proximal end 712a and the distal end 712b of the first needle shield 702. In various other examples, the teeth 822 may be configured to embed elsewhere in the first needle shield 702, including proximate the proximal end 712a or the distal end 712b.

As shown in FIG. 10, the cap 810 mechanically couples the first needle shield 702, second needle shield 704, and cap 810 together. The cap 810 is mechanically coupled with the second needle shield 704 when the annular grooves 818a, 818b disposed in the cap 810, receive annular ridges 828a, 828b disposed on the second needle shield 704. Additionally, the second needle shield 704 is coupled with the first needle shield 704 because the teeth 822 are embedded into the first needle shield 702 and the annular shoulder 716 coupled with the pocket 726. Accordingly, the first needle shield 702, second needle shield 704, and the cap 810 are mechanically coupled together.

When the first needle shield 702, second needle shield 704, and the cap 810 are coupled together, the force exerted on the rim 812 is distributed to the first needle shield 704 at least at the annular shoulder 716 and via the teeth 822. As a result, the even distribution of the force reduces deformation in the first needle shield 702. Since the first needle shield 702 doesn't deform, the force needed to remove the needle shield remains low (e.g., approximately 5-10 N).

In at least some of the embodiments of a needle shield assembly described herein, the combination of a first needle shield and a second needle shield, and additionally, in certain instances, a cap, may inhibit deformations in the first needle shield during removal of the needle shield assembly from a syringe and/or at other times. As a result, the first needle shield may be less likely to deform in a manner that increases frictional forces between the first needle shield and a needle of the syringe. Thus, at least some of the disclosed embodiments may reduce the force(s) necessary to remove the needle shield assembly from a syringe. Moreover, in at least some instances, feature(s) described herein in connection with one embodiment may be combined with feature(s) of one or more other embodiments described herein.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-a4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1 / c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Patent No. 7,153,507); Tysabri® (natalizumab, anti-a4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-a531 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716;

NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib),(2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/Mab Thera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1 x IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTER (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD3 BITER (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19 x CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor viII (EGFRvlll) BiTER (bispecific T cell engager) molecule. In some embodiments, the drug delivery device may contain or be used with AMG 673 or another product containing a half-life extended (HLE) anti-CD33 x anti-CD3 BITER (bispecific T cell engager) construct. In some embodiments, the drug delivery device may contain or be used with AMG 701 or another product containing a half-life extended (HLE) anti-B-cell maturation antigen (BCMA) x anti-CD3 BITER (bispecific T cell engager) construct. In some embodiments, the drug delivery device may contain or be used with AMG 757 or another product containing a half-life extended (HLE) anti-delta-like ligand 3 (DLL3) x anti-CD3 BiTE® (bispecific T cell engager) construct. In some embodiments, the drug delivery device may contain or be used with AMG 910 or another product containing a half-life extended (HLE) epithelial cell tight junction protein claudin 18.2 x CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A needle shield assembly, comprising:
a first needle shield at least partially made of a first material and having a proximal end and a distal end, the proximal end of the first needle shield comprising a cavity; and
a second needle shield at least partially made of a second material, the second needle shield mechanically coupled with at least the proximal end of the first needle shield,
wherein the second material is harder than the first material.

2. The needle shield assembly of claim 1, wherein the first needle shield comprises an annular shoulder and the second needle shield is mechanically coupled with the first needle shield by at least partially abutting the annular shoulder.

3. The needle shield assembly of claim 1, wherein the second needle shield abuts the proximal end of the first needle shield.

4. The needle shield assembly of claim 1, wherein at least a portion of the second needle shield overlays and/or is radially outward of at least a portion of the annular shoulder of the first needle shield.

5. The needle shield assembly of claim 1, wherein the second needle shield is overmolded on the first needle shield.

6. The needle shield assembly of claim 1, wherein the second needle shield comprises teeth, wherein the teeth are at least partially embedded in the first needle shield.

7. The needle shield assembly of claim 6, wherein the teeth are either (a) embedded in the first needle shield at approximately a midpoint between the proximal end and the distal end of the first needle shield, or (b) embedded in the distal end of the first needle shield.

8. (canceled)

9. A needle shield assembly, comprising:
a first needle shield having a proximal end and a distal end;
a second needle shield having a flexible distal portion and a rigid proximal portion; and
a cap configured to at least partially surround at least one of the first needle shield and the second needle shield and press the flexible distal portion of the second needle shield against the first needle shield.

10. The needle shield assembly of claim 9, wherein the second needle shield further comprises teeth coupled with the flexible distal portion.

11. The needle shield assembly of claim 10, wherein the teeth are at least partially embedded in the first needle shield when the cap at least partially surrounds the second needle shield.

12. The needle shield assembly of claim 11, wherein the teeth are disposed either (a) at a distal end of the flexible distal portion of the second needle shield, or (b) adjacent a mechanical connection between the flexible distal portion and the rigid portion of the second needle shield.

13. (canceled)

14. The needle shield assembly of claim 9, wherein the first needle shield further includes an annular shoulder and the second needle shield is mechanically coupled with the first needle by at least partially abutting the annular shoulder.

15. The needle shield assembly of claim 14, wherein the annular shoulder is disposed proximate the proximal end of the first needle shield.

16. The needle shield assembly of claim 9, wherein (a), (b), (c), and/or (d):
(a) the second needle shield abuts the proximal end of the first needle shield,
(b) the second needle shield comprises an annular ridge, the cap comprises an annular groove, and the annular ridge is configured to engage the annular groove to mechanically couple the second needle shield and the cap,
(c) the cap is configured to mechanically couple the first needle shield, second needle shield, and cap together, and/or
(d) the second needle shield is overmolded on the first needle shield.

17-19. (canceled)

20. A method of assembly, comprising:
providing a first needle shield at least partially made of a first material and having a proximal end and a distal end; and
mechanically coupling a second needle shield to the first needle shield, the second needle shield at least partially made of a second material, the second material being harder than the first material.

21. The method of assembly of claim 20, further comprising at least partially inserting the first needle shield and the second needle shield into a cap.

22. The method of assembly of claim 21, further comprising mechanically coupling the cap with the second needle shield, wherein mechanically coupling the cap with the second needle shield optionally comprises disposing at least one annular ridge of the second needle shield in at least one annular groove formed in the cap.

23. (canceled)

24. The method of assembly of claim 21, wherein the second needle shield includes a flexible distal portion and the cap presses the flexible distal portion of the second needle shield against the first needle shield, wherein the flexible distal portion optionally comprises at least one tooth and wherein mechanically coupling the cap with the second needle shield comprises pressing the flexible distal portion of the second needle shield against the first needle shield such that the at least one tooth is at least partially embedded within the first needle shield.

25. (canceled)

26. The method of assembly of claim 20, further comprising overmolding the second needle shield on the first needle shield.

27. The method of assembly of claim 20, wherein mechanically coupling the second needle shield to the first needle shield comprises disposing an annular shoulder of the first needle shield at least partially in a pocket of the second needle shield.
